(19) United States
(12) Patent Application Publication
(10) Pub. No.: US 2023/0419728 A1
(43) Pub. Date: Dec. 28, 2023

(54) SYSTEMS AND METHODS OF USING AUDIO AND STACKED COMPUTER VISION ALGORITHMS IN USER ENVIRONMENT AND USER BEHAVIOR DETECTION

(71) Applicant: EMED LABS, LLC, Miami, FL (US)

(72) Inventors: Sam Miller Miller, Hollywood, FL (US); Brian Schwab, Miami, FL (US); Zachary Carl Nienstedt, Wilton Manors, FL (US)

(21) Appl. No.: 18/341,756

(22) Filed: Jun. 26, 2023

Related U.S. Application Data
(60) Provisional application No. 63/366,983, filed on Jun. 24, 2022.
(60) Provisional application No. 63/357,298, filed on Jun. 30, 2022.

(51) Int. Cl. G06V 40/20; G06V 10/74; G06V 10/764; G16H 50/20

(52) U.S. Cl. CPC G06V 40/20; G06V 10/761; G06V 10/764; G16H 50/20; G06V 2201/03

(57) ABSTRACT

Systems and methods of using audio and stacked computer vision algorithms in user environment and user behavior detection are disclosed herein. In some embodiments, a ground truth stress test may be used to ensure a high quality and accuracy of a video feed captured of a user performing a diagnostic test. A set of stacked computer vision algorithms may be applied to the video feed in order to extract from the video feed a user behavior exhibited while the user performed the diagnostic test, evaluate a plurality of markers associated with the exhibited user behavior, and generate a marker profile for the behavior. The marker profile can be compared against a database to classify the behavior as a type of anomalous behavior. In some embodiments, pre-recorded audio utterances can also be selected and played to the user performing the diagnostic test for guidance purposes.

### CROSS-REFERENCE TO RELATED APPLICATIONS

This application claims the benefit of U.S. Provisional Patent Application No. 63/366,983, entitled “METHODS AND SYSTEMS FOR DELIVERING PROCTOR INFORMATION,” filed Jun. 24, 2022, the contents of which are incorporated by reference herein in their entirety. This application also claims the benefit of U.S. Provisional Patent Application No. 63/357,298, entitled “SYSTEMS AND METHODS FOR DETECTING USER ENVIRONMENTS USING STACKED HEURISTIC COMPUTER VISION ALGORITHMS,” filed Jun. 30, 2022, the contents of which are incorporated by reference herein in their entirety.

### TECHNICAL FIELD

The embodiments of the disclosure generally relate to remote medical diagnostic testing and testing platforms. More specifically, the embodiments relate to systems and methods for using audio recordings and computer vision algorithms in the context of virtually proctored, self-administered diagnostic testing. For instance, stacked computer vision algorithms can be used to detect and evaluate the user testing environment and user behavior during performance of the diagnostic test, and audio recordings can also be used to provide instructions to the user based on the user's actions (e.g., as determined through computer vision).

### BACKGROUND

Remote or at-home healthcare testing and diagnostics can solve or alleviate some problems associated with in-person testing. For example, health insurance may not be required, travel to a testing site is avoided, and tests can be completed at a testing user's convenience. However, remote or at-home testing introduces various additional logistical and technical issues, such as relying on a user's interpretation of test results. The use of telehealth technologies can alleviate some of these issues by allowing for long distance patient and health provider contact, such as via a user or patient's personal user device (e.g., a smartphone, tablet laptop, personal computer, or other device). For example, a user or patient can interact with a remotely located medical care provider using live video, audio, or text-based chat through the personal user device in order to receive guidance and/or oversight of the testing procedures remotely.

However, providing real-time, synchronous monitoring and guidance via live video or audio can be especially overwhelming, time-consuming, and inefficient when done on a 1:1 basis. This can be the case when there is a large quantity of remote or at-home diagnostic testing being performed simultaneously that requires monitoring. This can be especially problematic in some instances, such as when adherence to diagnostic test procedure and test result authenticity is supremely important (e.g., a diagnostic test for the presence of an illegal substance).

Thus, there exists a need to provide proctors and medical care providers with the technology to efficiently manage and supervise a large number of remote diagnostic tests, such as by offloading some of the burden by automating some of the monitoring and guidance tasks.

### SUMMARY

For purposes of this summary, certain aspects, advantages, and novel features are described herein. It is to be understood that not necessarily all such advantages may be achieved in accordance with any particular embodiment. Thus, for example, those skilled in the art will recognize the disclosures herein may be embodied or carried out in a manner that achieves one or more advantages taught herein without necessarily achieving other advantages as may be taught or suggested herein.

All of the embodiments described herein are intended to be within the scope of the present disclosure. These and other embodiments will be readily apparent to those skilled in the art from the following detailed description, having reference to the attached figures. The invention is not intended to be limited to any particular disclosed embodiment or embodiments.

The embodiments of the disclosure generally relate to systems and methods for using audio and stacked computer vision algorithms in user environment and user behavior detection. In some embodiments, a ground truth stress test may be used to ensure a high quality and accuracy of a video feed captured of a user performing a diagnostic test. A set of stacked computer vision algorithms may be applied to the video feed in order to extract from the video feed a user behavior exhibited while the user performed the diagnostic test, evaluate a plurality of markers associated with the exhibited user behavior, and generate a marker profile for the behavior. The marker profile can be compared against a database to classify the behavior as a type of anomalous behavior. In some embodiments, pre-recorded audio utterances can also be selected and played to the user performing the diagnostic test for guidance purposes.

A user may undergo (e.g., self-administer) a medical diagnostic test that the user may select from a medical diagnostic test kit container. The user may administer the medical diagnostic test with the aid of a remote medical diagnostic testing platform. A user device (e.g., a smartphone, table, laptop, etc.), of which the remote medical diagnostic testing platform may be implemented on or accessed by, may scan the medical diagnostic testing kit container or medical diagnostic test using computer vision to recognize the type of test being used. In some embodiments, using computer vision, the system may determine whether a proctor can be used for guidance or is required to guide the user through the user's administration of the medical diagnostic test. Once the system determines whether a proctor can be used for guidance through the administration of the medical diagnostic test, the system may direct the user to the appropriate platform accordingly. In cases where a proctor is not necessary, the system may provide the user with other forms of instruction and guidance, such as written, video, or augmented reality guidance, among others.

A user may initiate the use of a system to assist in administer a health or diagnostic test. The system may administer a robot ground truth stress test. The system may administer heuristics on a prerecorded or live video feed. The system may identify anomalous behavior presented by the user. The system may identify heuristics associated with the anomalous behavior identified. The system may analyze a database and select heuristics that may be similar to the heuristics identified. The system may process archived data and output human or augmented labels, which may be used in a feedback cycle. The system may utilize a feedback cycle or loop to train the heuristics and collect additional data.

### BRIEF DESCRIPTION OF THE DRAWINGS

These and other features, aspects, and advantages of the disclosure are described with reference to drawings of certain embodiments, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1 illustrates a system diagram of a telehealth proctoring platform that can be used to proctor, monitor, and manage patients over the course of a medical diagnostic test or a medical treatment plan.

FIG. 2 illustrates a block diagram illustrating an example protocol or method for a computer vision system that detects user environments and behavior using stacked heuristic computer vision algorithms, in accordance with embodiments disclosed herein.

FIG. 3 illustrates a block diagram of an example protocol or method for a telehealth proctoring platform that allows proctors to conduct virtual proctoring sessions using pre-recorded utterances, in accordance with embodiments disclosed herein.

FIG. 4 presents a block diagram illustrating an embodiment of a computer hardware system configured to run software for implementing one or more embodiments of the systems and methods disclosed herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

For example, in some instances, a system implementing a stacked heuristic computer vision algorithm may be used to detect characteristics of a user's environment or scene. The environment or scene detected may be the setting or surroundings of which the user may be administering the health or diagnostic test within. In some instances, the system may detect the type of health or diagnostic test the user may administer. In some instances, the system may detect the type of health or diagnostic test the user may administer by detecting of identifying the box or container of which the health or diagnostic test is in. In some instances, in detecting or identifying the type of health or diagnostic test the user may administer, the system may compare the type of test box or container identified to a list of tests supported by a health or diagnostic testing platform. Such platform may include a proctor platform.

In some instances, the system may correctly detect and identify the type of test box or container to determine whether the type of test is supported by the platform. In some instances, the test box or container may be held by the user, located on a chair that may be the same or different color as the test box, located on a bed chair that may be the same or different color as the test box, in mid-air, etc. In some instances, the test box may blend in with its surroundings. In some instances, the system may be able to differentiate between various types of boxes or container upon determining there is more than one type of box or container within the user environment. In some instances, the system may track the movement of the box or container throughout the user environment. In some instances, the system may implement a feedback loop which may train the computer vision algorithm to improve the identification or detection capability of the system.

In some instances, the user may initiate the use of the system. In some instances, the system may initially implement a robot ground truth stress test. The stress test may adapt to the user's environment. For example, the stress test may vary across lighting conditions, camera angles, scene occlusions, etc. The system may implement the stress test to initially assess the environment the user may administer the health or diagnostic test within.

In some instances, the system may administer heuristics on a prerecorded or live video feed. In some instances, the system may administer the heuristics using augmented reality or virtual reality. In some instances, the heuristics may be included in a continuous integration suite.

In some instances, the system may identify anomalous behavior issued by the user. For example, the system may include human or a robot (e.g., A Human in the Loop), which may be configured to identify or mark anomalous behavior issued by a user. For example, anomalous behavior issued by a user may include flailing arms, throwing items, fast movements, etc.

In some instances, from the identified anomalous behavior, the system may further identify heuristics associated with the anomalous behavior identified. For example, such heuristics identified can include markers or indicators such as size of cluster, color profile, camera exposure, certainty or direction of a deep neural network (DNN) tensor, etc. In some instances, the system may analyze a database, which may include previously identified heuristics and user data. The system may be configured to select and extract images from the database that may compare or represent a similar heuristic marker profile as the heuristic identified.

In some instances, the system may label the data within the database. In labeling the data, the system may output a human augmented or artificial intelligence augmented label. Such labels may be used in a feedback loop, which may train the heuristics associated with anomalous behavior and collect additional data continuously.

Anomalous behavior or scenes may have negative implications on the performance of a system during administration of a test. This limitation may cause a user to become frustrated during administration of the test. This may lead to inaccurate test results. Accordingly, it may be beneficial to provide a system that can identify and target anomalous behavior and train itself to improve its performance upon the identified problematic scenarios.

In some instances, users may access an online proctoring platform while taking an at-home or self-administered medical diagnostic test. The proctoring platform may be configured to provide guidance to the user to facilitate taking the medical diagnostic test, to verify that the medical diagnostic test is properly administered, and/or to verify the results of the medical diagnostic test. One or more portions of the medical diagnostic test can be administered to the user by a live proctor of the proctoring platform. For example, the user and the proctor can be connected via an audio or video call that allows the proctor to communicate with the user. In some instances, the proctor has an approved script that is read to the user over to provide information to the user about various steps of the testing process (e.g., sample collection process, results interpretation process, etc.).

Over the course of a shift, the live proctor may read the same script many times. While the initial readings of the script may have intonation, inflection, and other perceived conversational or friendly qualities, the script may eventually become memorized and may be recited in a flat, monotone voice with a cadence that is rushed or that does not follow the user's actions.

To improve the user experience and to reduce performance requirements of the proctors, pre-recorded utterances (e.g., specific chunks of the script) in the proctor's voice may be played at appropriate times for delivery of instruction to the user. The pre-recorded utterances may include portions or the entirety of the standard at-home diagnostic test script as well as other versions of the script (e.g., expedited/redacted versions of the script for experienced users, common supplemental instructions needed for inexperienced users, common responses to user questions or comments, etc.).

In some embodiments, the pre-recorded utterances may be collected after the proctor has completed a training course. This timing may advantageously occur after the proctor has been familiarized to understand and easily read the script and before the proctor experiences fatigue from re-reading the script many times. The recording can be repeated until a mistake-free, high-quality, personable version of the reading is obtained.

In some embodiments, delivery of a specific utterance to a user is triggered by the proctor. For example, a button tied to an utterance may be pressed such that the utterance is played (e.g., to the user only or to both the user and the proctor).

In some cases, the proctor may not wish to hear their voice recording repeatedly throughout the day. A text version of the utterance may be displayed to the proctor as a reminder of what the user is hearing so that the proctor can easily keep track of the user experience. In some embodiments, a visual indicator, light, graphic, sound, or other notification may be provided to the proctor when the utterance begins and finishes so that the proctor knows when to move to the next step.

In some cases, the proctor may be presented with multiple pre-recorded utterance options to select from. These may be provided based on commonly uttered phrases associated with certain steps in the testing process.

For example, during the sample collecting phase, it may be common for a proctor to provide the standard swabbing instructions as well as to correct the user if they are not inserting the nose swab properly, if they need to switch the swab to the other side, or if they did not complete the number of swabs required by the test, etc. Each of these common instructional phrases may be displayed for easy access as the proctor and the user enter this phase of the test experience.

A full library of pre-recorded utterances may also be made available to the proctor so that relevant messages can be delivered to the user with minimal time delay so that the communication feels like live communication.

Alternatively, the proctor may select to turn on their microphone and speak to the user directly. Because the pre-recorded utterances are in the proctor's voice, the interjection of a live (e.g., not pre-recorded) voice will not be noticeable by the user.

In some embodiments, a computer vision algorithm may receive video data from the user device and may be trained to identify certain actions that are associated with certain proctor utterances. When the algorithm identifies that the user is performing certain actions, the associated recording of the proctor's voice may be delivered to the user to correct, instruct, educate, or otherwise communicate to the user.

The utterances may be placed in a playlist to walk the user through the testing experience automatically based on the detected actions. This may allow the proctor to take a more managerial approach to supervising test sessions. The proctor may be able to watch multiple sessions at once and only interject if the user needs help or if the user is doing something incorrectly. The interjection is not noticeable to the user because the pre-recorded voice matches the live voice for a coherent experience.

The described approach advantageously provides the proctor with tools to guide a user through a test experience using their optimal reading of the test for improved consistency and quality while reducing the cognitive load and stress associated with reading a script many times. Additionally, the approach allows for personalization and customization of the script, timing, and supplemental information provided so that the user still receives a personalized experience.

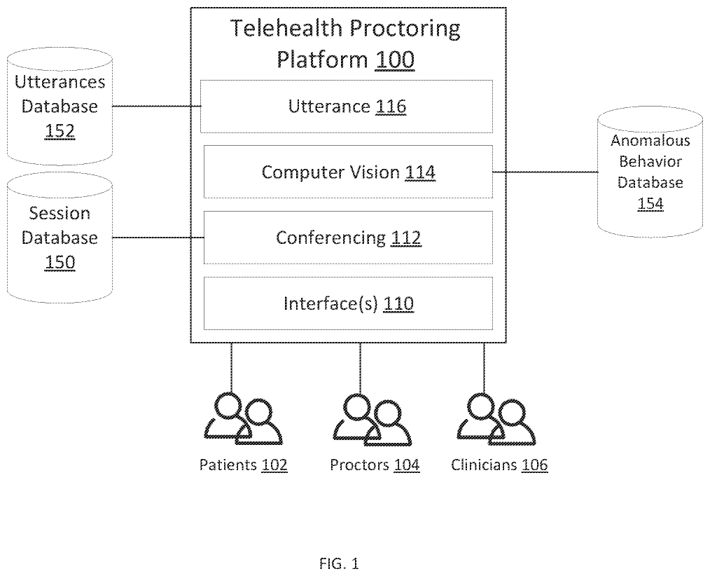

FIG. 1 illustrates a system diagram of a telehealth proctoring platform that can be used to proctor, monitor, and manage patients that are performing a diagnostic test or participating in a medical treatment plan. More specifically, it illustrates a system diagram of a telehealth proctoring platform 100 that can be used to proctor, monitor, and manage patients 102 performing a medical diagnostic test (e.g., a lateral flow test to detect the presence of COVID-19 or an illegal substance in a sample), participating in a medical treatment plan (e.g., a weight loss program involving self-administering of weight loss medication), and so forth.

It should be noted that functionality of the telehealth proctoring platform 100 may be shown as components, and that the various components of the telehealth proctoring platform 100 are illustrated and described in a manner to facilitate ease of understanding. In practice, one or more of the components may be optional, used together, or combined. Furthermore, one or more of the components may be separately located (e.g., with a third-party or at an endpoint) or their corresponding functionality may be performed at various locations. For example, the interfaces 110 may include user interfaces displayed to a patient through a website or web-based application (with the user interface data provided by a server on the backend), or alternatively, the user interfaces could be displayed by an application running on the patient's user device (and that application may or may not be part of the telehealth proctoring platform 100).

It also should be noted that the term “users” may sometimes refer to the patients 102 (who are proactively using the telehealth proctoring platform 100), especially in the context of self-administering medical diagnostic tests or participating in a medical treatment plan. However, the term “users” may also refer to any of the patients 102, proctors 104, and/or clinicians 106 (who could all be considered users in the literal sense since they all interact with the telehealth proctoring platform 100).

The patients 102, proctors 104, and/or clinicians 106 may be able to interact with the telehealth proctoring platform 100 through one or more interfaces 110 associated with the telehealth proctoring platform 100. These interfaces 110 may include various user interfaces and/or application programming interfaces (APIs) depending on the implementation of the telehealth proctoring platform 100. For example, in some embodiments, one or more of the patients 102, proctors 104, and/or clinicians 106 may access the telehealth proctoring platform 100 via their user device by accessing an application installed on their user device or a web-based application, which will provide various user interfaces that can be interact with. In some embodiments, one or more of the patients 102, proctors 104, and clinicians 106 may access an application installed on their user device or a web-based application, and that application may communicate with the telehealth proctoring platform 100 via an API.

Examples of the patients 102 may include any person that is self-administering a diagnostic test, such as a lateral flow test, and any person receiving or registered to receive medical treatment as part of a medical treatment plan, such as a weight loss program. A lateral flow test is a simple device intended to detect the presence or absence of a target analyte in a liquid sample, and it is widely used in medical diagnostics in the home, at the point of care, and in the laboratory. For example, it can be used to detect specific target molecules, such as molecules associated with pathogens and diseases, gene expression or biomarkers in humans (e.g., hormones and other proteins), chemicals or toxins, and so forth. It can test a variety of samples, like urine, blood, saliva, sweat, serum, and other fluids. Lateral flow tests can also be used in other contexts beside medical diagnostics, such as food safety and environmental monitoring. Some non-limiting examples of specific diagnostic tests that the patients 102 may use the telehealth proctoring platform 100 for may include COVID-19 diagnostic tests or drug diagnostic tests for detecting the presence of a drug (e.g., illegal or prescription drugs) in a sample.

Examples of the proctors 104 may include medical professionals (e.g., physician, nurse, nutritionist, health coach, and/or the like) that can provide instructions or real-time guidance to a patient that is performing a diagnostic test or self-administering medication. In addition, the proctors 104 can monitor performance of the diagnostic test for adherence to proper test procedure and to ensure test result authenticity; provide suggestions or interpretations of diagnostic test results; and monitor the patient's progress during a medical treatment plan. For instance, a proctor can be a medical professional that virtually meets with a patient to go over instructions for a lateral flow test to detect COVID-19 and then assists the patient with interpreting the results of the lateral flow test. As another example, the proctor could virtually meet with a patient to go over instructions for a medical weight loss program.

In some embodiments, the proctors 104 may be tasked with flagging anomalous user behaviors exhibited in the video feeds of patients 102 performing diagnostic tests. In some embodiments, the telehealth proctoring platform 100 may include a computer vision system that can use computer vision algorithms (such as stacked heuristic algorithms) in order to process and review video feeds and detect any anomalous user behaviors, which may make it easier for the proctors 104 to effectively supervise large numbers of patients 102. In some embodiments, the proctors 104 may be able to provide instructions or real-time guidance to a patient that is performing a diagnostic test through the use of pre-recorded audio scripts or utterances, which may also make it easier for the proctors 104 to effectively supervise large numbers of patients 102.

Examples of the clinicians 106 may be any doctor that has contact with, and direct responsibility for, a patient and is capable of approving or modifying a patient's medical treatment plan.

In some embodiments, the telehealth proctoring platform 100 can include a conferencing system or module 112. In some embodiments, the conferencing module 112 can be configured to connect a patient 102 and a proctor 104 in a telehealth or virtual proctoring session. In some embodiments, the conferencing module 112 can be configured to connect the patient 102 and the proctor 104 via a video conferencing session, such as via live video (e.g., over the Internet of cellular communication network). In some embodiments, the conferencing module 112 can be configured to facilitate video calls, audio calls and/or telemedicine calls. In some embodiments, the patient 102 may access the conferencing module 112 via their user device (not shown), and the proctor 104 may access the conferencing module 112 via their user device (e.g., a proctor device, not shown).

In some embodiments, the conferencing module 112 may be configured to establish a live, virtual proctoring session between a patient and a proctor. For example, it may enable a patient 102 to provide to a proctor 104 a live video feed of the patient 102 performing a diagnostic test. A patient may be assigned to a specific proctor or a group of proctors in advance (e.g., to a particular medical professional or group of medical professionals), or the patient may be assigned to one of the proctors 104 based on availability (e.g., who is available when the patient initiates a proctoring session), and/or based on personal considerations (e.g., the patient's sex, gender, age, co-morbidities, dietary preferences, and so forth).

Virtual proctoring sessions may be scheduled (e.g., at regular intervals as part of a medical treatment plan) or initiated by the patient on-demand (e.g., based on performance of a diagnostic test). The flexibility (e.g., remote, scheduled or on-demand, and seamlessly managed by the telehealth proctoring platform 100) of the virtual proctoring sessions and the manner that they are implemented in the telehealth proctoring platform 100 may provide numerous benefits, and a non-inclusive list is provided. First, they allow the telehealth proctoring platform 100 to enable greater interaction between the patient and a medical professional. The virtual proctoring sessions can be used to easily track patient compliance with a medical treatment plan or testing procedure by monitoring or tracking a patient's self-administration of a procedure, treatment, or medication.

In some embodiments, recordings (e.g., video recordings) associated with a proctoring session may be saved in a database such as a session database 150, and data associated with a proctoring session may also be saved in the session database 150

In some embodiments, the telehealth proctoring platform 100 may include a computer vision system or module 114. The computer vision system 114 may utilize various image processing techniques and/or computer vision techniques to analyze the video conferencing feed, video recording, and/or still images captured from a proctoring session (either in real-time or on historical data, e.g., saved to session database 150). In some embodiments, the computer vision system 114 may perform processing and analysis by various AI, machine learning, and/or heuristic techniques.

In some embodiments, the computer vision system 114 may be associated with an anomalous behavior database 154. In some embodiments, the anomalous behavior database 154 may define patterns, templates, or profiles for different kinds of anomalous user behavior, which the computer vision system 114 may be able to use for comparison purposes to detect anomalous user behavior in video feeds. For example, different types of anomalous behavior may be defined using a heuristic marker profile, which represents the set of heuristic markers and their value ranges that best characterize that anomalous behavior.

In some embodiments, the telehealth proctoring platform 100 may include an utterances system or module 116, which may be associated with an utterance database 152. More specifically, the proctors 104 may record utterances (e.g., specific chunks of the script used to provide guidance/instruction on a diagnostic test) in their own voice and save them to the utterance database 152. The utterances module 116 may allow the proctor to select pre-recorded utterances in their own voice to be played at appropriate times for delivery of instruction to the user. During a proctoring session, the utterances module 116 may determine which pre-recorded utterance options are available at a specific time or step in a diagnostic testing process (e.g., there may be commonly uttered phrases associated with certain steps in the testing process), then present the proctor with those multiple pre-recorded utterance options to select from (e.g., via a graphical user interface displayed on the proctor's device). The proctor may be able to select a particular utterance which will be played to the user to provide additional guidance and instruction while the user carries out the diagnostic test.

Thus, it can be understood that the telehealth proctoring platform 100 described herein can offer many advantages. For example, the patients 102 may not need to travel for periodic check-ins. In some embodiments, the patients 102 can speak with proctors 104, clinicians 106, or other medical professionals (e.g., physician, nutritionist, health coach, and/or the like) on demand or with short notice (for example, a same-day appointment, an appointment within a few days, and so forth). In some embodiments, the telehealth proctoring platform 100 can be used to enable proctors 104, clinicians 106, or other medical professionals to provide guidance or instructions to a patient, such as instructions for performing a diagnostic test. In some embodiments, the patients 102 may be able to conduct some forms of testing at home under proctored supervision, such as checking blood glucose levels or for the presence of COVID-19.

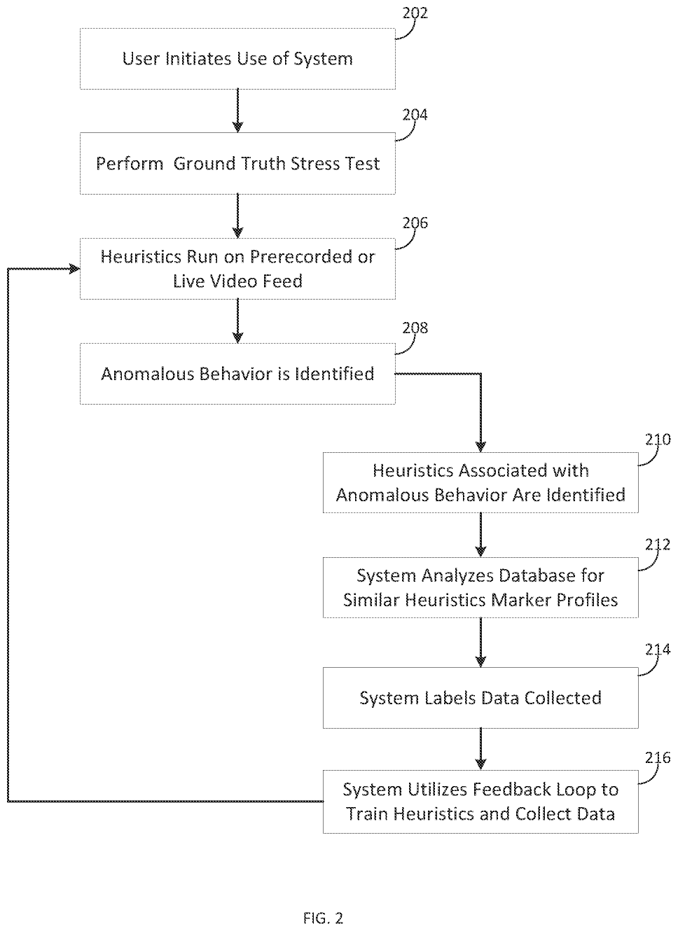

FIG. 2 illustrates a block diagram of an example protocol or method for a computer vision system that detects user environments and behavior using stacked heuristic computer vision algorithms. The method can be implemented, for example, using one or more components of the system shown in FIG. 1.

Accuracy of diagnostic or health test results interpretation can be important. Proctored or certified remote diagnostic testing may rely on cameras or sensors of a user device to relay image to proctors or other systems for their viewing. Test results interpreted by a user, under guidance of a proctor or not, can introduce human error. Accordingly, it may be beneficial to implement a computer vision system that can detect and evaluate surrounding user environments, in order to ensure sufficient quality and accuracy of images received from the user device so that computer vision-based results interpretation can be used to interpret test results more accurately. In some embodiments, the computer vision systems disclosed herein may use stacked heuristic computer vision algorithms in order to detect and evaluate surrounding user environments.

Anomalous user behavior may have negative implications on the performance of a system during administration of a diagnostic test. This limitation may cause a user to become frustrated during administration of the diagnostic test. This may lead to inaccurate test results. Accordingly, it may be beneficial to implement a computer vision system that can identify anomalous user behavior and train itself to improve its performance whenever there is anomalous user behavior. In some embodiments, the computer vision systems disclosed herein may use stacked heuristic computer vision algorithms in order to detect anomalous user behavior.

At block 202, a user (e.g., a patient) may initiate use of the computer vision system to assist in the administering of a diagnostic test (e.g., a lateral flow test), procedure, or medication. For example, a user may want to perform a medical diagnostic test using a medical diagnostic test kit container and with the aid of a telehealth proctoring platform or a remote medical diagnostic testing platform (e.g., to receive remote guidance during a virtual proctoring session).

In some embodiments, the user may be able to input information about the medical diagnostic test (e.g., the test type) or procedure into a user device (e.g., a smartphone, table, laptop, etc.), such as into an application installed on, or accessed by, the user device. This information can be provided to the telehealth proctoring platform and/or used by the system to directly determine the type of diagnostic test or procedure involved.

In other embodiments, computer vision algorithms can be used to recognize the diagnostic test being used, the medication being administered, and so forth. For instance, computer vision may be able to recognize a test or medication based on one or more associated features, such as the container, labelling, contents, or machine-readable codes. For example, the test may have a machine-readable code (e.g., a barcode, a QR code, a serial number, etc.) that may identify the medical diagnostic test or medical diagnostic test kit container. In some embodiments, the user may be able to scan the medical diagnostic testing kit container or medical diagnostic test with the user device (e.g., with a camera on the user device), such as using an application installed on, or accessed by, the user device. Thus, the computer vision system may be able to recognize the type of test being used from an image or video captured by the user device.

In some embodiments, the determined type of test or procedure can be used to further determine whether a proctor can be used for guidance or is required to guide the user through the user's administration of the medical diagnostic test or procedure. Once the computer vision system determines whether a proctor can be used for guidance through the administration of the medical diagnostic test, the system may direct the user to the appropriate platform accordingly (e.g., to set up a virtual proctoring session through a telehealth proctoring platform). In cases where a proctor is not necessary, the system may provide the user with other forms of instruction and guidance, such as written, video, or augmented reality guidance, among others.

In some embodiments, a recorded or live video feed of the user performing the medical diagnostic test or procedure may need to be captured for review. These diagnostic tests or procedures may require the user to perform different steps or actions throughout the administration of the test session. This can be done using an application installed on, or accessed by, the user device and one or more cameras of the user device. For example, the user may have to capture a video feed of themselves performing a medical diagnostic test with their user device because that medical diagnostic test may require live proctor supervision (e.g., in a virtual proctoring session over the telehealth proctoring platform) and/or algorithmic review for anomalous behaviors.

However, there may be a need to ensure that the video feed of the user will be of sufficient quality and accuracy. The video feed needs be clear for a proctor to supervise the procedure and for computer vision algorithms to process the video to detect anomalous behaviors. Additionally, the color, lighting, etc., in the video may need to adjusted and corrected for (due to differences associated with a user's test administration environment, user device, etc.) in order to maximize the accuracy of manual or automated results interpretation (e.g., such as when the results of the diagnostic test requires comparisons to a color gradient).

Thus, at block 204, the computer vision system may administer a ground truth stress test in order to assess the image/video quality and accuracy. The computer vision system may be able to make corrections and adjustments to the video based on the determinations, and it may also be able to determine when/if the image/video quality and accuracy are insufficient (e.g., to notify the user). The stress test may adapt to the user's environment. For example, the stress test may vary across lighting conditions, camera angles, scene occlusions, etc. The computer vision system may implement the stress test to initially assess the environment that the user may administer the diagnostic test within. Additional discussion of this process is provided in U.S. patent application Ser. No. 18/129,744, entitled “SYSTEMS AND METHODS FOR ENSURING AND VERIFYING REMOTE HEALTH OR DIAGNOSTIC TEST QUALITY,” which is hereby incorporated by reference in its entirety.

As an example, a medical diagnostic testing kit container or medical diagnostic test may be include or be associated with a reference card that contains test identifying elements and/or other elements that can be used in test interpretation, such as a code, a results strip, a color gradient, etc. The code may include a QR code, NFC tags, image recognizers (e.g., images, patterns, etc., that can be used to identify the test), etc. The user may be directed to capture an image of the reference card (e.g., with the camera of their user device) so that the results strip may be seen in the image on the front of the reference card. Various regions of the reference card can be used to preprocess the image and verify the image quality. For example, an image can be preprocessed prior to results interpretation.

In some instances, for example, to ensure, verify, or improve test quality, the quality of the image captured may be assessed. To assess the image quality and/or preprocess the image, the computer vision system may scan or interpret the code within the captured image. In some embodiments, the code can include a unique ID, which may be different for each reference card manufactured and can allow each reference card to be individually recognized or individually identified. In some instances, the unique ID can be used to track the diagnostic or health test throughout the testing process, which may mitigate risk of fraud or error. Additional information may be contained within the unique ID, or the unique ID can be used to retrieve additional information from a database or other data store. For example, a manufacturing lot number, an expiration date of the test, the test type, the test version, and so forth can be determined, either directly from the code or by querying a database or other data store. Much of the information contained within the unique ID or linked to the unique ID (e.g., in a database or other data store) can be used to further differentiate the test from others. For example, the manufacturing lot number can be used to track manufacturing defects. Similarly, for example, the expiration date can be used to validate the efficacy of the test prior to administration of the test. As another example, the unique ID can be used to determine whether a test with the same unique ID has already been taken, which can indicate an attempt to commit fraud and/or to reuse a test. As another example, the test type and test version can be found in a database to determine where various reference card features, test strip identifiers or lines, and so forth should appear.

Additionally, in some instances, for example, the computer vision system may align the captured image to match a template. Alignment of the image to the template allows the system to be able to register which pixel regions to look at for the various features on the reference card. In some instances, the system can implement feature matching to align the image, which may be an effective method to align the image. For example, the features of the reference card can be used to obtain a geometric transform that aligns the captured image with the template, for example by minimizing a mean squared error, mean absolute error, and so forth. In some embodiments, the captured image can be considered to be aligned when the difference between the captured image and the template is below a threshold value. In some instances, after alignment of the image and the template, fixed pixel range addresses can be used to access the other information on the reference card. This may also improve further processing, manual or automatic, by ensuring every test image can be standardized in orientation, resolution, and so forth.

Additionally, in some instances, for example, the computer vision system can correct the color of the captured image. The captured image of the reference card can include a color reference chart or gradient, which may include printed known colors. By measuring the colors of one or more areas of the captured image, the system can adjust the image to represent “true” or “reference” color(s) for the test strip. In some instances, if the color of the image is not corrected, the test strip elements or test strip stripes may have a different color profile than may be expected due to the type of lighting in the environment where the image was captured, the white balance of the camera, and so forth. In some instances, restoring the “true” or “reference” color can improve the accuracy of manual or automatic interpretation of results by normalizing the appearance of images. In some embodiments, some or all of these corrections and adjustments can be applied to the video feed of the user performing the diagnostic test or procedure.

Additionally, in some instances, for example, the system can check whether an image is of a sufficient quality to detect a test result at or above the limit of detection. For example, a reference card can include graduated color strips. The system may evaluate the graduated color stripes that may be located on the test strip or reference card to determine a detection threshold. In some instances, the lightest or faintest graduated color stripe can be printed on the reference card at an intensity that may be slightly below the desired detection threshold for the actual test strip. By treating these lines as “results lines” and attempting to detect the “results lines,” the system can assess whether the image has sufficient dynamic range, contrast, brightness, noise floor, etc., to be able to detect a “weak positive” result that may be on the actual test strip. The system may use region of interest, pixel region averaging, color isolation, deep learning, etc., to determine whether the image is of sufficient quality to detect a test result at or above the limit of detection. This may be useful in maximizing the accuracy of manual or automated results interpretation.

In some embodiments, the system may determine the sharpness of the captured image. In some instances, the camera used to capture the image may utilize an autofocus feature of the camera to sharpen the image to be captured. However, such features can still result in blurry or fuzzy images due to, for example, poor lighting, focus on a different object in the field of view of the camera, and so forth. The system can help to ensure the image captured has sufficient sharpness. For example, the system can check various regions of the image (e.g., QR code, company logo, fiducials, edges, etc.) to measure blur. The system may reject images that are too blurry. For example, the system may reject an image in which features that are expected to be sharp are spread over too many pixels, when transitions from one color to another occur over too many pixels, and so forth. For example, if a logo is printed in black on a white card, a sharp transition from black to white can be expected at the edges of the logo. If the transition is not sudden, this may indicate that the image is too blurry. In some instances, a blurry image can cause edges to expand and may impact the appearance of results lines. In some instances, the interpretation of the results after administration of a diagnostic test may be improved by both manual and automatic means when the system can ensure a sharp image.

In some embodiments, an image may pass the quality check upon the completion of the previous corrections and checkpoints, such as scan code, align image, correct image color, check detection threshold, check image sharpness, etc. In some instances, corrections or checkpoints may be completed simultaneously. The system may determine at any point along the image quality verification process that the image may not be of sufficient quality and direct a user to collect an additional image. In some embodiments, the system can direct the user to change the setup of the image capture, for example by increasing lighting, moving closer to the object to be imaged (e.g., a reference card), and so forth.

In some instances, if the image quality is not sufficient, the user may be guided to change the setup for improved image quality. For example, the user may be guided to improve the user's environment so that the next image captured may be of more sufficient quality. Various variables may affect the adequacy of the user's environment such as brightness, lighting location, shadows, dirty camera lens, etc. For example, more light in the room may reduce noise and improve image quality, a bright light source in front of or directly behind the reference card may degrade the image, an overhead light or indirect lighting may enhance the image, shadows from nearby objects may also affect image quality, a dirty camera lens may degrade or blur the image, etc. In some instances, the system may recognize various types of lighting environments and may assess the image and intelligently suggest the best alteration of the environment to the user, along with graphics or animated instructions. In some instances, the system may prompt the user with instructions such as, for example, “Please turn on an additional light or move to a brighter place,” “Please turn the test around so that the nearest window is behind you,” “Please clear the area around the test of any large objects,” “Please gently wipe off your camera lens,” etc.

Once it is determined that the images/video produced by the user device in the user's test administration environment are of sufficient quality and accuracy, a live video feed of the user performing the diagnostic test or procedure may be captured with the user device (e.g., for a virtual proctoring session). The live video feed can be transmitted to the telehealth proctoring platform and/or a proctor's user device. The proctor may be able to remotely provide the user with guidance and instructions while the user performs various steps or actions associated with the diagnostic test or procedure. The proctor may also watch to ensure that the user is adhering to proper test procedure and to ensure the test results are authentic. In some embodiments, the video feed may be recorded and saved with the telehealth proctoring platform for later retrieval and analysis (e.g., via the computer vison system).

At block 206 and block 208, the computer vision system may apply one or more heuristic algorithms to the prerecorded or live video feed of the user performing the diagnostic test or procedure in order to identify and mark any anomalous behavior that the user exhibited during the diagnostic test or procedure. More specifically, a plurality of heuristic algorithms (e.g., stacked heuristic computer vision algorithms) can be carefully selected and used together to carry out various computer vision tasks or techniques for acquiring, processing, analyzing and understanding the images in the live or prerecorded video feed. Examples of such tasks include object recognition, identification, detection, and tracking; pose estimation; facial recognition; shape recognition; and human activity recognition. These techniques can also be used to extract higher-dimensional data from the video and obtain a high level of contextual understanding of the user's activities (e.g., movements, actions, etc.). The stacked heuristic algorithms may further be used to analyze the user's activities for anomalous behavior (e.g., by comparing the user's activities to various patterns associated with anomalous behavior).

In some embodiments, these heuristic algorithms may be applied to the live video feed of the user in real-time (e.g., during the virtual proctoring session) in order to identify and mark the user's anomalous behavior. Although the proctor may be able to closely watch the video to make sure that the user is adhering to proper test procedure and that test results are authentic (e.g., there is no anomalous user behavior), this may not be feasible for a human in practice. The proctor can easily miss out on identifying anomalous user behavior that occurs quickly or is designed to trick humans (e.g., the user is actively trying to tamper with the test results). Furthermore, the telehealth proctoring platform may be designed to allow a proctor to simultaneously supervising many diagnostic tests or procedures at a time, so the proctor's attention may be stretched thin.

The objective of a heuristic algorithm is to produce a solution in a reasonable time frame that is good enough for solving the problem at hand. This solution may not be the best of all the solutions to this problem, or it may simply approximate the exact solution, but it is still valuable because finding it does not require a prohibitively long time. Accordingly, multiple heuristic algorithms can also be used together to tackle related computer vision tasks without requiring a prohibitively long time. Thus, stacked heuristic algorithms may be well-suited for the role of constantly monitoring live video feeds for anomalous behavior, so that if anomalous user behavior is identified, it can be flagged and immediately brought to the proctor's attention (e.g., during the virtual proctoring session itself). The proctor can then take remedial action, such as by asking the user to perform steps of the diagnostic test or procedure again.

These heuristic algorithms may also be applied to prerecorded videos of the user to identify and mark the user's anomalous behavior, which can be useful for identifying anomalous behavior in older saved recordings, for improving the heuristic algorithms, for analyzing user behavior for tests or procedures that do not require live proctoring, and so forth. In some instances, the computer vision system may administer the stacked heuristics algorithms using augmented reality or virtual reality. In some instances, the heuristics algorithms may be included in a continuous integration suite.

In some embodiments, the computer vision system may utilize the stacked heuristic algorithms to detect characteristics of a user's environment or scene (e.g., the setting or surroundings of which the user may be administering the diagnostic test within). The computer vision system may utilize the stacked heuristic algorithms to detect the type of diagnostic test the user may administer. In some instances, the computer vision system may utilize the stacked heuristic algorithms to detect the type of diagnostic test the user may administer by detecting or identifying the box or container that the diagnostic test is in. In some instances, the computer vision system may utilize the stacked heuristic algorithms to compare the type of test box or container identified to a list of tests supported by a diagnostic testing platform or telehealth proctoring platform.

In some instances, the computer vision system may utilize the stacked heuristic algorithms to correctly detect and identify the type of test box or container to determine whether the type of test is supported by the platform. In some instances, the test box or container may be held by the user, located on a chair that may be the same or different color as the test box, located on a bed chair that may be the same or different color as the test box, in mid-air, etc. In some instances, the test box may blend in with its surroundings. In some instances, the computer vision system may utilize the stacked heuristic algorithms to be able to differentiate between various types of boxes or containers upon determining that there is more than one type of box or container within the user environment. In some instances, the computer vision system may utilize the stacked heuristic algorithms to track the movement of the box or container throughout the user environment. In some instances, the computer vision system may implement a feedback loop for training and improving the stacked heuristic algorithms, thereby improving the identification or detection capability of the system.

In some embodiments, the computer vision system may involve an anomalous behavior database. In some embodiments, the anomalous behavior database may define patterns, templates, or profiles for different kinds of anomalous user behavior. Some examples of different anomalous user behavior include flailing arms, throwing items, fast movements, etc. In some embodiments, different types of anomalous behavior in the database may be associated with, and identified using, different sets of heuristic markers or indicators (e.g., metrics). Examples of various heuristic markers or indicators include size of cluster, color profile, camera exposure, certainty or direction of a deep neural network (DNN) tensor, etc. Taking it a step further, each type of anomalous behavior may be defined by a heuristic marker profile, which represents the set of heuristic markers and their value ranges that best characterize that anomalous behavior.

Thus, in some embodiments, the stacked heuristic algorithms may be used to compare the user's activities in the video against the various patterns of anomalous behavior defined in the anomalous behavior database, and a user activity can be classified as a particular type of anomalous behavior if a similar match is found. More specifically, in some embodiments, the stacked heuristic algorithms may be used to monitor the user's activities in the videos, evaluate the different heuristic markers (e.g., determine their values) for the activities, generate heuristic marker profiles for the activities, and compare those generated heuristic marker profiles with heuristic marker profiles in the anomalous behavior database to find a similar match (e.g., within a similarity threshold).

In some embodiments, the anomalous behavior database may initially need to be compiled (e.g., through human interaction/observation and/or supervised learning). For instance, the telehealth proctoring platform may include humans (e.g., proctors) or human-in-the-loop (HITL) tasked with identifying, flagging, and labeling anomalous user behavior that arises in the videos of the virtual proctor sessions. Researchers and developers of the telehealth proctoring platform may gather the data (e.g., the video segments) associated with many instances of a particular anomalous behavior (e.g., flailing arms) once there are enough samples. The heuristic markers (e.g., used with the stacked heuristic algorithms) can be evaluated for all these instances and used to generate a heuristic marker profile that best captures that anomalous behavior (e.g., flailing arms) while reducing false positives. In this manner, heuristic marker profiles can be generated for other types of anomalous behavior for inclusion into the anomalous behavior database.

In some embodiments, there may be a feedback loop that can be used to add to and improve the anomalous behavior database. In some cases, this may also involve human interaction/observation and/or supervised learning. For instance, at block 208, the computer vision system may include humans (e.g., proctors) or human-in-the-loop (HITL) tasked with identifying, flagging, and labeling anomalous user behavior that arises in the videos of the virtual proctor sessions. In one instance, a proctor could identify an anomalous user behavior (e.g., fast movement) that is not yet defined in the anomalous behavior database.

At block 210, the computer vision system may identify heuristic criteria associated with the identified anomalous behavior, such as evaluating a set of heuristic markers (e.g., used by the stacked heuristic algorithms), like size of cluster, color profile, camera exposure, certainty or direction of a deep neural network (DNN) tensor, etc. In other words, the computer vision system may put together a loosely-fitting heuristic marker profile for the identified anomalous behavior.

At block 212, the computing vision system may analyze a database, such as a session database, which may include recorded video data from previous virtual proctor sessions. In embodiments, the database may also include previously identified or evaluated heuristics from the video data. The computer vision system may be configured to select and extract video or images from the database that may have a similar heuristic marker profile as the identified anomalous behavior.

At block 214, the computing vision system may process the selected/extracted data by labeling some or all of it. In some embodiments, the system may output a human augmented or artificial intelligence augmented label. For example, all the extracted video data that exhibit the desired type of anomalous behavior may be labeled for association with that type of anomalous behavior.

At block 216, the labeled data may be used in a feedback loop to further train and improve the heuristics associated with anomalous behavior and collect additional data continuously. For instance, in some embodiments, the labeled data associated with a particular type of anomalous behavior can be used to generate a heuristic marker profile for that behavior to be added to the anomalous behavior database so that the behavior can be detected with the stacked heuristic algorithms.

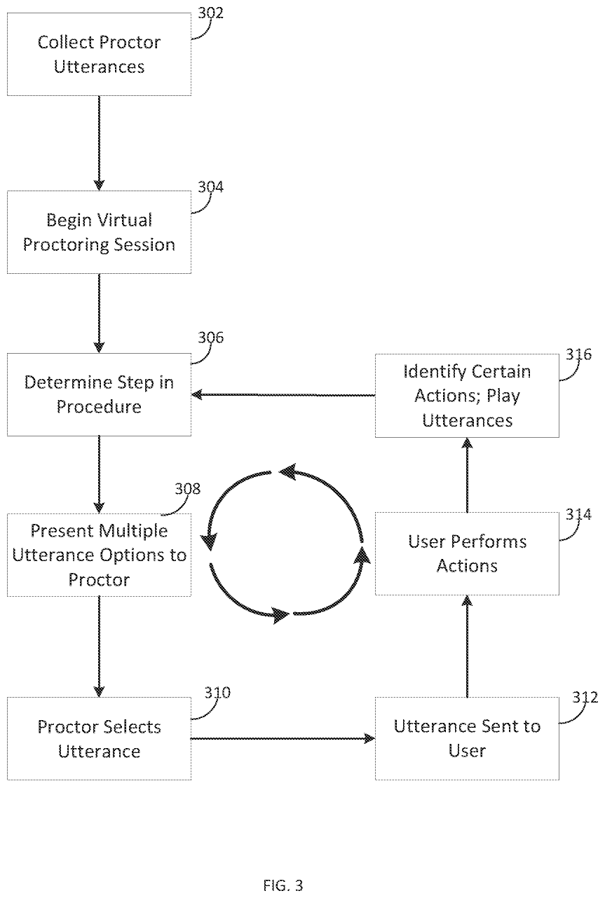

FIG. 3 illustrates a block diagram of an example protocol or method for a telehealth proctoring platform that allows proctors to conduct virtual proctoring sessions using pre-recorded utterances, thereby improving the efficiency and scalability of the telehealth proctoring platform. The method can be implemented, for example, using one or more components of the system shown in FIG. 1.

In some instances, users may access a telehealth proctoring platform (e.g., online, such as through a web-based application or an installed application on their device) while taking an at-home or self-administered medical diagnostic test, self-administering a medical procedure or medication, or checking in as part of a medical treatment plan. For instance, the proctoring platform may be configured to provide guidance to the user to facilitate taking the medical diagnostic test, to verify that the medical diagnostic test is properly administered, and/or to verify the results of the medical diagnostic test. Guidance for one or more portions of the medical diagnostic test can be provided by a live proctor through the proctoring platform. For example, the user and the proctor can be connected via an audio or video call that allows the proctor to communicate with the user.

In some instances, the proctor has an approved script that is read to the user over to provide information to the user about various steps of the testing process that may be associated with a particular diagnostic test (e.g., sample collection process, results interpretation process, etc.). However, the proctor may be responsible for supervising many users and many diagnostic testing procedures. Over the course of a shift, the live proctor may read the same script many times. While the initial readings of the script may have intonation, inflection, and other perceived conversational or friendly qualities, the script may eventually become memorized and may be recited in a flat, monotone voice with a cadence that is rushed or that does not follow the user's actions.

Furthermore, in some cases, a proctor may even be responsible for supervising many users that are performing different types of diagnostic tests and procedures, which may require the use of many different scripts (e.g., each script may only be associated with a particular type of diagnostic test or procedure). Sometimes the proctor may have to supervised multiple users simultaneously. All of this can be difficult for the proctor to keep track of, and it can increase the likelihood that the proctor will read the wrong script for the test or procedure, read the script at the wrong step, or even lose track of their place in a script entirely (e.g., due to their attention going back-and-forth).

To improve the user experience and to reduce performance requirements of the proctors, pre-recorded utterances (e.g., specific chunks of the script) in the proctor's voice may be played at appropriate times for delivery of instruction to the user. The pre-recorded utterances may include portions or the entirety of the standard at-home diagnostic test script as well as other versions of the script (e.g., expedited/redacted versions of the script for experienced users, common supplemental instructions needed for inexperienced users, common responses to user questions or comments, etc.).

At block 302, pre-recorded utterances may be collected from the proctor. In some embodiments, the pre-recorded utterances may be collected after the proctor has completed a training course. This timing may advantageously occur after the proctor has been familiarized to understand and easily read the script and before the proctor experiences fatigue from re-reading the script many times. The recording can be repeated until a mistake-free, high-quality, personable version of the reading is obtained. The available pool of pre-recorded utterances associated with the proctor may be continually updated and added to over time.

In some embodiments, pre-recorded utterances may be collected as long as there is no ongoing virtual proctor session for the proctor. Thus, at block 304, once a virtual proctoring session starts (e.g., the telehealth proctoring platform initiates a video call between the proctor and a user, during which the proctor provides guidance to the user to perform a diagnostic test), the proctor may not be able to add to their pre-recorded utterances.

At block 306, the telehealth proctoring platform may determine the current step of the testing process. In some embodiments, the proctor and/or the user may indicate what the current step is. In some embodiments, the computer vision system and/or the stacked heuristic algorithms may be able to automatically determine the current step of the testing process based on context and the type of diagnostic test being used.

At block 308, the proctor may be presented with multiple pre-recorded utterance options to select from (e.g., via a graphical user interface displayed on the proctor's device). The options may be based on the current step of the testing process; there may be commonly uttered phrases associated with certain steps in the testing process.

For example, during the sample collecting phase, it may be common for a proctor to provide the standard swabbing instructions as well as to correct the user if they are not inserting the nose swab properly, if they need to switch the swab to the other side, or if they did not complete the number of swabs required by the test, etc. Each of these common instructional phrases may be displayed for easy access as the proctor and the user enter this phase of the test experience.

In some embodiments, a full library of pre-recorded utterances may also be made available to the proctor so that relevant messages can be delivered to the user with minimal time delay so that the communication feels like live communication.

At block 310, the proctor may select a specific utterance (e.g., within the graphical user interface displayed on the proctor's device) from among the options to deliver to the user. For example, a button tied to an utterance may be pressed such that the utterance is played (e.g., to the user only or to both the user and the proctor). In some embodiments, the proctor may alternatively select to turn on their microphone and speak to the user directly instead of using a pre-recorded utterance. Because the pre-recorded utterances are in the proctor's voice, the interjection of a live (e.g., not pre-recorded) voice will not be noticeable by the user.

At block 312, a selected utterance may be played to the user (e.g., on the user's device) or to both the user and the proctor. In some cases, the proctor may not wish to hear their own voice recording repeatedly throughout the day. A text version of the utterance may be displayed to the proctor as a reminder of what the user is hearing so that the proctor can easily keep track of the user experience. In some embodiments, a visual indicator, light, graphic, sound, or other notification may be provided to the proctor when the utterance begins and finishes so that the proctor knows when to move to the next step.

At block 314, the user may perform actions in accordance with the current step of the procedure and the instructions received form the proctor (e.g., via the selected utterance). For example, if the proctor used a pre-recorded utterance to send standard swabbing instructions to the user during the sample collection phase, the user may proceed to insert the nose swab by following the instructions.

At block 316, in some embodiments, while the user performs actions, a computer vision algorithm may receive video data from the user device and may be trained to identify certain actions that are associated with certain proctor utterances. When the algorithm identifies that the user is performing certain actions, the associated recording of the proctor's voice may be delivered to the user to correct, instruct, educate, or otherwise communicate to the user. In some embodiments, the utterances may be placed in a playlist to walk the user through the testing experience automatically based on the detected actions. In some embodiments, the computer vision algorithm may be a computer vision system that employs stacked heuristic algorithms in order to identify the certain user actions. This may allow the proctor to take a more managerial approach to supervising test sessions. The proctor may be able to watch multiple sessions at once and only interject if the user needs help or if the user is doing something incorrectly. The interjection is not noticeable to the user because the pre-recorded voice matches the live voice for a coherent experience.

At this point, the procedure may cycle through blocks 306, 308, 310, 312, 314, 316 (and then back to block 306) repeatedly until all the steps in the procedure have been performed. However, the ordering does not necessarily have to be strict. For instance, while the user performs an action at block 314, the proctor may notice that the user is incorrectly performing that action and preemptively select an utterance at block 310 that informs the user of their mistake or the correct action. The utterance can then be sent to the user device to be played to the user.

The described approach advantageously provides the proctor with tools to guide a user through a test experience using their optimal reading of the test for improved consistency and quality while reducing the cognitive load and stress associated with reading a script many times. Additionally, the approach allows for personalization and customization of the script, timing, and supplemental information provided so that the user still receives a personalized experience.

### Computer Systems

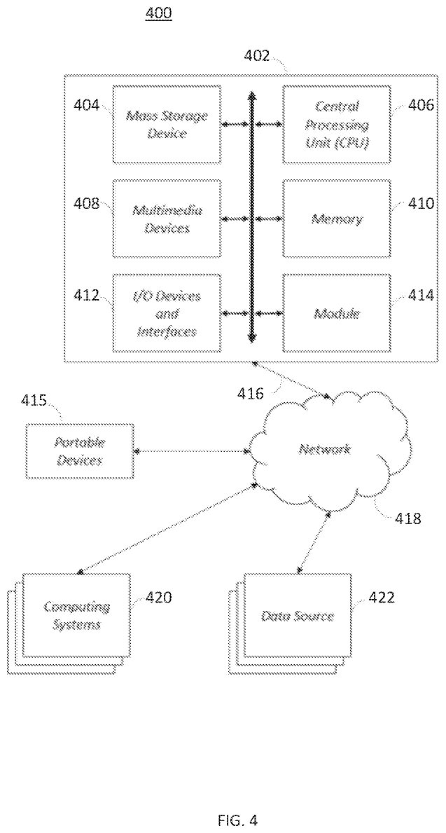

FIG. 4 is a block diagram depicting an embodiment of a computer hardware system configured to run software for implementing one or more embodiments of the health testing and diagnostic systems, methods, and devices disclosed herein. The example computer system 402 is in communication with one or more computing systems 420 and/or one or more data sources 422 via one or more networks 418. While FIG. 4 illustrates an embodiment of a computing system 402, it is recognized that the functionality provided for in the components and modules of computer system 402 may be combined into fewer components and modules, or further separated into additional components and modules.

The computer system 402 can comprise a module 414 that carries out the functions, methods, acts, and/or processes described herein. The module 414 is executed on the computer system 402 by a central processing unit 406 discussed further below.

In general, the word “module,” as used herein, refers to logic embodied in hardware or firmware or to a collection of software instructions, having entry and exit points. Modules are written in a program language, such as JAVA, C or C++, PYPHON or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 402 includes one or more processing units (CPU) 406, which may comprise a microprocessor. The computer system 402 further includes a physical memory 410, such as random-access memory (RAM) for temporary storage of information, a read only memory (ROM) for permanent storage of information, and a mass storage device 404, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 402 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 402 includes one or more input/output (I/O) devices and interfaces 412, such as a keyboard, mouse, touch pad, and printer. The I/O devices and interfaces 412 can include one or more display devices, such as a monitor, which allows the visual presentation of data to a user. More particularly, a display device provides for the presentation of GUIs as application software data, and multi-media presentations, for example. The I/O devices and interfaces 412 can also provide a communications interface to various external devices. The computer system 402 may comprise one or more multi-media devices 408, such as speakers, video cards, graphics accelerators, and microphones, for example.

The computer system 402 may run on a variety of computing devices, such as a server, a Windows server, a Structure Query Language server, a Unix Server, a personal computer, a laptop computer, and so forth. In other embodiments, the computer system 402 may run on a cluster computer system, a mainframe computer system and/or other computing system suitable for controlling and/or communicating with large databases, performing high volume transaction processing, and generating reports from large databases. The computing system 402 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 402 illustrated in FIG. 4 is coupled to a network 418, such as a LAN, WAN, or the Internet via a communication link 416 (wired, wireless, or a combination thereof). Network 418 communicates with various computing devices and/or other electronic devices. Network 418 is communicating with one or more computing systems 420 and one or more data sources 422. The module 414 may access or may be accessed by computing systems 420 and/or data sources 422 through a web-enabled user access point. Connections may be a direct physical connection, a virtual connection, and other connection type. The web-enabled user access point may comprise a browser module that uses text, graphics, audio, video, and other media to present data and to allow interaction with data via the network 418.

Access to the module 414 of the computer system 402 by computing systems 420 and/or by data sources 422 may be through a web-enabled user access point such as the computing systems' 420 or data source's 422 personal computer, cellular phone, smartphone, laptop, tablet computer, e-reader device, audio player, or another device capable of connecting to the network 418. Such a device may have a browser module that is implemented as a module that uses text, graphics, audio, video, and other media to present data and to allow interaction with data via the network 418.

The output module may be implemented as a combination of an all-points addressable display such as a cathode ray tube (CRT), a liquid crystal display (LCD), a plasma display, or other types and/or combinations of displays. The output module may be implemented to communicate with input devices 412 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 402 may comprise a physical or logical connection established between a remote microprocessor and a mainframe host computer for the express purpose of uploading, downloading, or viewing interactive data and databases on-line in real time. The remote microprocessor may be operated by an entity operating the computer system 402, including the client server systems or the main server system, an/or may be operated by one or more of the data sources 422 and/or one or more of the computing systems 420. In some embodiments, terminal emulation software may be used on the microprocessor for participating in the micro-mainframe link.

In some embodiments, computing systems 420 who are internal to an entity operating the computer system 402 may access the module 414 internally as an application or process run by the CPU 406.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 402 may include one or more internal and/or external data sources (for example, data sources 422). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 402 may also access one or more databases 422. The databases 422 may be stored in a database or data repository. The computer system 402 may access the one or more databases 422 through a network 418 or may directly access the database or data repository through I/O devices and interfaces 412. The data repository storing the one or more databases 422 may reside within the computer system 402.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

We claim:

1. A computer-implemented method for processing a video feed of a user performing a diagnostic test using a set of stacked computer vision algorithms, the method comprising:
receiving, from a user device, the video feed of the user performing the diagnostic test, wherein the video feed was evaluated for quality and accuracy using a ground truth stress test applied to an image captured by the user device, wherein the captured image is of a reference card associated with the diagnostic test;
applying the set of stacked computer vision algorithms to the video feed to extract, from the video feed, a user behavior exhibited while the user performed the diagnostic test;
evaluating a plurality of markers associated with the exhibited user behavior based on the set of stacked computer vision algorithms;
generating a marker profile for the exhibited user behavior based on the plurality of markers associated with the exhibited user behavior;
comparing the marker profile for the exhibited user behavior against marker profiles in an anomalous behavior database;
identifying a similar marker profile in the anomalous behavior database, wherein the similar marker profile is associated with a type of anomalous user behavior; and
classifying the exhibited user behavior under the type of anomalous user behavior.

2. The computer-implemented method of claim 1, wherein the set of stacked computer vision algorithms comprises a plurality of heuristic computer vision algorithms.

3. The computer-implemented method of claim 1, wherein the plurality of markers are a plurality of heuristic markers.

4. The computer-implemented method of claim 1, wherein the marker profile is a heuristic marker profile.

5. The computer-implemented method of claim 1, further comprising:
flagging an instance of anomalous user behavior;
evaluating a second plurality of markers associated with the instance of anomalous user behavior;
generating a second marker profile for the instance of anomalous user behavior based on the second plurality of markers;
searching a database for video data having marker profiles similar to the second marker profile; and
extracting the video data having similar marker profiles from the database.

6. The computer-implemented method of claim 1, wherein the reference card comprises a color reference chart for color correction.

7. The computer-implemented method of claim 1, wherein the ground truth stress test is configured to assess an environment that the user may perform the diagnostic test within.

8. A non-transient computer readable medium containing program instructions for causing a computer to perform a method for processing a video feed of a user performing a diagnostic test using a set of stacked computer vision algorithms, the method comprising:
receiving, from a user device, the video feed of the user performing the diagnostic test, wherein the video feed was evaluated for quality and accuracy using a ground truth stress test applied to an image captured by the user device, wherein the captured image is of a reference card associated with the diagnostic test;
applying the set of stacked computer vision algorithms to the video feed to extract, from the video feed, a user behavior exhibited while the user performed the diagnostic test;
evaluating a plurality of markers associated with the exhibited user behavior based on the set of stacked computer vision algorithms;
generating a marker profile for the exhibited user behavior based on the plurality of markers associated with the exhibited user behavior;
comparing the marker profile for the exhibited user behavior against marker profiles in an anomalous behavior database;
identifying a similar marker profile in the anomalous behavior database, wherein the similar marker profile is associated with a type of anomalous user behavior; and
classifying the exhibited user behavior under the type of anomalous user behavior.

9. The non-transient computer readable medium of claim 8, wherein the set of stacked computer vision algorithms comprises a plurality of heuristic computer vision algorithms.

10. The non-transient computer readable medium of claim 8, wherein the plurality of markers are a plurality of heuristic markers.

11. The non-transient computer readable medium of claim 8, wherein the marker profile is a heuristic marker profile.

12. The non-transient computer readable medium of claim 8, wherein the method performed by the computer further comprises:
flagging an instance of anomalous user behavior;
evaluating a second plurality of markers associated with the instance of anomalous user behavior;
generating a second marker profile for the instance of anomalous user behavior based on the second plurality of markers;
searching a database for video data having marker profiles similar to the second marker profile; and
extracting the video data having similar marker profiles from the database.

13. The non-transient computer readable medium of claim 8, wherein the reference card comprises a color reference chart for color correction.

14. The non-transient computer readable medium of claim 8, wherein the ground truth stress test is configured to assess an environment that the user may perform the diagnostic test within.
